(19) United States
(12) Patent Application Publication
(10) Pub. No.: US 2026/0207581 A1
(43) Pub. Date: Jul. 23, 2026

(54) Small Molecule Inhibitor for Inflammatory Pain Inhibition

(71) Applicant: University of Rochester, Rochester, NY (US)

(72) Inventors: Thomas Diekwisch, Rochester, NY (US); Xianghong Luan, Rochester, NY (US)

(21) Appl. No.: 19/137,058

(22) PCT Filed: Jan. 10, 2024
(86) PCT No.: PCT/US2024/011068

§ 371 Date: Jun. 9, 2025

Related U.S. Application Data
(60) Provisional application No. 63/479,237, filed on Jan. 10, 2023.

(51) Int. Cl. A61K 31/4725; A61K 9/00; A61K 9/06; A61K 9/50; A61K 9/51; A61P 19/02

(52) U.S. Cl. CPC A61K 31/4725; A61K 9/0019; A61K 9/06; A61K 9/5068; A61K 9/5169; A61K 9/5176; A61P 19/02

(57) ABSTRACT

Provided are compositions and methods for treating inflammatory pain. In some embodiments, the inflammatory pain is associated with a temporo-mandibular joint and muscle disorder (TMD).

### CROSS-REFERENCE TO RELATED APPLICATIONS

The present application claims priority to U.S. Provisional Application No. 63/479,237, filed on Jan. 10, 2023, the contents of which are hereby incorporated by reference in their entirety.

### BACKGROUND OF THE INVENTION

According to the National Institute for Dental and Craniofacial Research (NIDCR), the prevalence of temporomandibular joint and muscle disorders (TMD) is between 5% and 12%, and the annual cost associated with TMD treatment is approximately $4 billion. In a recent Consensus Study Report of the National Academies of Sciences, Engineering, and Medicine, the committee on temporomandibular disorders summarizes that TMDs are not only difficult to define but also difficult to treat (Bond et al. 2020). Chronic TMD pain is a debilitating condition, frequently associated with elevated levels of suicidal ideation, depression, and anxiety (Bertoli and de Leeuw 2016). Historically, patients with TMD were frequently subjected to orthodontic treatment, correction of malocclusion, or orthognathic surgery, resulting in little therapeutic success (Al-Moraissi et al. 2015, Shroff 2018). Early surgical replacement of the TMJ complex with Proplast-Teflon implants was marred by implant disintegration and continued foreign body giant cell presence even years after implant placement (Henry and Wolford 1993, Estabrooks et al. 1990). More recent approaches such as discectomy or disc replacement strategies have either failed to alleviate the symptoms or resulted in further TMJ degeneration (Hagandora and Almarza 2012). As a result, there is an enormous need for effective treatment modalities that will make a significant difference in the lives of patients suffering from chronic pain.

Pain is the most common symptom of TMDs, usually concentrated in masticatory muscles and/or temporomandibular joints (Furquim et al. 2015). A systematic review groups 45.3% of TMD patients into the myofascial pain category, 41.1% in a myofascial pain with limited opening category, and 30.1% into an arthralgia, osteoarthritis, and osteoarthrosis category (Manfredini et al. 2011). Others have estimated that the majority of TMD etiologies (Stengenga et al. 1989, Landesberg and Wadhwa 2013) are due to inflammatory joint degeneration (osteoarthritis). In support of an inflammation induced TMD etiology, inflammatory cytokines have been associated with TMDs and identified as biomarkers for chronic TMD pain (Slade et al. 2011). The present application focuses on inflammation-related changes in temporo-mandibular joint disease and on the development of viable treatment modalities for inflammatory TMDs. Inflammation occurs in temporo-mandibular joint (TMJ) tissues either due to systemic inflammatory arthritis (O'Connor et al. 2017) or following biomechanical stress as a result of trauma or intrinsic and/or extrinsic joint overload, resulting in synovitis, capsulitis, retrodiscitis, ligamentitis, and arthritis of the TMJ (Dijkgraaf et al. 1996a, b). “Despite investment in research directly and indirectly related to temporomandibular disorders (TMD)—most significantly in the field of orofacial pain—researchers have yet to unravel the etiologies and pathophysiologies of TMD”, states a current consensus report by the National Academies of Sciences (Bond et al. 2020).

As such, there is a need in the art for a method of treating TMDs and the pain associated therewith. The present invention addresses this long felt, but unmet, need.

### SUMMARY OF THE INVENTION

In various embodiments, the present disclosure provides a method of treating or reducing pain in a subject in need thereof, comprising administering to the subject a therapeutically effective amount of a composition comprising PFI-2 or a derivative, prodrug, or pharmaceutically acceptable salt thereof. In some embodiments, the pain is associated with one or more inflammatory diseases or disorders. In some embodiments, the one or more inflammatory disease or disorders are one or more temporo-mandibular joint and muscle disorders (TMDs). In some embodiments, the composition is injected directly into the temporo-mandibular joint (TMJ) or the surrounding tissue.

In some embodiments, the composition comprises one selected from the group consisting of a hydrogel, a nanoparticle, an exosome, and combinations thereof.

In some embodiments, the hydrogel is a collagen hydrogel or extracellular matrix (ECM) hydrogel. In some embodiments, the ECM hydrogel is a decellularized ECM (dECM) hydrogel. In some embodiments, the collagen hydrogel is a Type I collagen (Col I) hydrogel, with Col I at a concentration of about 10 mg/mL. In some embodiments, the concentration of PFI-2 in the hydrogel is about 20 μM.

In some embodiments, the nanoparticle is a Col I hydrogel nanoparticle or a dECM hydrogel nanoparticle.

In some embodiments, wherein the exosome is derived from a mesenchymal stem cell (MSC). In some embodiments, the MSC is a synovial MSC.

In some embodiments, the present disclosure provides a composition comprising PFI-2 or a derivative, prodrug, or pharmaceutically acceptable salt thereof and one or more selected from the group consisting of a hydrogel, a nanoparticle, an exosome, and combinations thereof. In some embodiments, the composition is formulated for injection.

In some embodiments, the hydrogel is a collagen hydrogel or an ECM hydrogel. In some embodiments, the collagen hydrogel is a Col I hydrogel. In some embodiments, the ECM hydrogel is a dECM hydrogel.

In some embodiments, the nanoparticle is a Col I hydrogel nanoparticle or a dECM hydrogel nanoparticle.

In some embodiments, the exosome is derived from an MSC. In some embodiments, the MSC is a synovial MSC.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1, comprising FIG. 1A through FIG. 1J, depicts representative results demonstrating that PFI-2 treatment reversed the degeneration of TMJ tissues, inhibited inflammation and downregulated inflammatory gene expression. FIG. 1A-FIG. 1C. Rat heads 48 hours after saline injection (FIG. 1A) vs. CFA injection (FIG. 1B) or CFA plus PFI-Disc hydrogel injection (FIG. 1C) to illustrate regional swelling. The distance between the two ear tragi was increased by 10% in the CFA treatment group (FIG. 1B) and reduced to the original distance in the CFA-PFI treatment group (FIG. 1C). FIG. 1D-FIG. 1F. 3D-reconstruction of uCT images of condyles and temporal fossae from saline (FIG. 1A), CFA (FIG. 1B) or CFA plus PFI-2 (FIG. 1C) injection. Note the decrease in condylar bone volume and the increase in the joint space between temporal fossa and condyle (FIG. 1E). The defects were rescued by PFI-2 treatment (FIG. 1F). FIG. 1G-FIG. 1I. H&E staining of paraffin-embedded TMJ tissues. In the CFA injection group, the TMJ disc was thickened and fused with the condyle. Thickness of the fibrous and proliferative cell zones was increased while the pre-hypertrophic and hypertrophic zones were decreased in thickness (FIG. 1H). PFI-2 treatment resulted in the detachment of the TMJ disc and condyle that were fused in the inflammation model, reduced the thickness of the fibrous and proliferative cell layers and rescued the structure and function of fibrocartilage in the TMJ (FIG. 1I). FIG. 1J. qRT-PCR analysis of inflammatory genes and YAP pathway members in control and TMD tissues.

FIG. 2, comprising FIG. 2A through FIG. 2K, depicts representative results demonstrating that PFI-2 treatment reversed inflammation-induced degeneration of TMJ matrix and modulated YAP1 expression in TMDs. FIG. 2A. Masson trichrome images revealed fibrous adhesion of TMJ disc and condylar surface following inflammation while PFI-2 de-attached the TMJ disc from the condyle. FIG. 2B. Pico Sirius red staining showed yellow-green thin collagen fibers in the control group compared to dark red, thick fibers in CFA-treated group. PFI-2 treatment resulted in a conversion to light red, thinner fibers in the TMD disc. FIG. 2C. YAP1 immunolocalization in TMJ discs. Inflammation increased YAP1 expression when compared to controls while PFI-2 treatment translocated YAP into nuclei. FIG. 2J and FIG. 2K. qRT-PCR analysis of matrix protein and adhesion molecule expression in TMJ tissues in control (SV), CFA-treated (CV) and CFA-PFI-treated (CP) groups. Note that inflammation upregulated the expression of matrix proteins and adhesion molecules except Collagen 2. PFI-2 treatment reversed the upregulation.

FIG. 3, comprising FIG. 3A through FIG. 3P, depicts representative results demonstrating that PFI-2 treatment rescued inflammation-induced inhibition of chondrocyte maturation and returned YAP expression in the condylar growth plate. (FIG. 3A, FIG. 3D, FIG. 3G, FIG. 3J, and FIG. 3M) are saline-injected controls (SV); (FIG. 3B, FIG. 3E, FIG. 3H, FIG. 3K, and FIG. 3N) are TMJ tissues exposed to inflammatory conditions following CFA-injection (CV); and (FIG. 3C, FIG. 3F, FIG. 3I, FIG. 3L, and FIG. 30) were injected with PFI-2 following CFA induction of inflammatory conditions. (FIG. 3A-FIG. 3C) H&E staining, (FIG. 3D-FIG. 3F) Safranin O staining, (FIG. 3G-FIG. 3I) Immunoreactions detecting YAP, (FIG. 3J-FIG. 3L) Cell proliferation assay based on PCNA staining, (FIG. 3M-FIG. 30) Immuno-reactions for the cartilage differentiation marker Sox 9. (FIG. 3P) summarizes results of RT-PCR reactions measuring relative gene expression of Collagens 2 and 10, Mmp13 metalloproteinase, Runx2 bone transcription factor, and Sox 9. Note the increased thickness of the proliferative cell layer, a displacement of the chondrocytic cell layer in inferior direction (FIG. 3E, FIG. 3H, and FIG. 3K), and retention of Sox9 in the chondrocytic cell layer in the inflammation group. There was also a significant reduction in overall Sox9 expression (FIG. 3P) and a reduction of Sox9 in the hypotrophic cell layer (FIG. 3N) of the CFA-treated group. The inflammation-induced retardation of chondrocyte differentiation was reversed in the PFI-2 treated group.

FIG. 4, comprising FIG. 4A through FIG. 4F, depicts representative changes in pain behavior and neuropeptide expression in our inflammatory TMD model. FIG. 4A and FIG. 4B, Meal duration experiment. CFA treatment prolonged the food uptake time (FIG. 4B vs. FIG. 4A). FIG. 4C and FIG. 4D. Immunostaining of CGRP protein in the synovial membrane of control (FIG. 4C) and CFA treated (FIG. 4D) groups. CGRP was localized in synovial cells and neurons and was increased in the CFA treated group (FIG. 4D). FIG. 4E. qRT-PCR analysis of neuropeptides and their receptor expression in saline-treated (SV), CFA-treated (CV) or CFA-PFI-treated (CP) TMJ tissues. The expression of CGRP, NPY and TAC was upregulated in the CV group while the changes were almost reduced to normal levels in the CP group. FIG. 4F. ELISA measurement of CGRP expression in TMJ tissues. The amount of CGRP was significantly increased at day 7 followed by a gradual decrease until day 42 after CFA injection.

FIG. 5, comprising FIG. 5A through FIG. 5N, depicts representative results demonstrating that the methyltransferase SETD7 modulates YAP localization, expression and function in RSSCs under inflammatory condition. FIG. 5A-FIG. 5C, and FIG. 5G. Relative changes in SETD7 nuclear localization. TNF-α treatment increased SETD7 nuclear localization as well as expression in the cytoplasm (FIG. 5B and FIG. 5G). However, the SETD7 inhibitor PFI-2 blocked the effect of TNF-α on SETD7 nuclear localization (FIG. 5C and FIG. 5G). D-F, H. Changes in SETD7 localization as revealed by immunofluorescence. TNF-α treatment decreased YAP nuclear localization (FIG. 5E and FIG. 5H) while SETD7 knockdown using SETD7 siRNA promoted YAP nuclear translocation and increased the number of YAP positive nuclei (FIG. 5F and FIG. 5H). FIG. 5I. qRT-PCR analysis of IL-18 and IL-6 expression after knockdown of P65, SETD7 and YAP. P65 and SETD7 knockdown reduced while YAP knockdown increased the expression of inflammatory cytokines (FIG. 5I). FIG. 5J and FIG. 5K. Western blot analysis of protein methylation state in whole cell lysates (FIG. 5J) and YAP expression in the nucleus (FIG. 5K). TNF-αtreatment enhanced global protein methylation levels while PFI-2 inhibited this effect (FIG. 5J). Furthermore, TNF-α treatment reduced YAP nuclear translocation, while SETD7 siRNA and PFI-2 increased YAP nuclear translocation (FIG. 5J). FIG. 5L and FIG. 5M. Chip assays of YAP binding to the SOX9 promoter TEAM binding site demonstrated that knockdown of YAP1 reduced the enrichment of YAP1 on the SOX9 promoter (FIG. 5L) while the expression of SOX9 was increased (FIG. 5M) as demonstrated by RT-PCR. FIG. 5N. Changes in selective neuropeptide expression upon YAP knockdown in RSSCs. Transfection of YAP siRNA resulted in an upregulation of CGRP, Galanin and TAC expression, while NPY was downregulated.

FIG. 6, comprising FIG. 6A through FIG. 6N, depicts representative results that PFI-2 rescued TNFα-induced inflammatory phenotypes and gene dysregulation in RSSCs and TMJ explants. FIG. 6A-FIG. 6C. BrdU labeling to correlate the effect of modulation of inflammation with proliferation. TNFa treatment decreased the number of BrdU positive proliferating cells (FIG. 6B), while PFI-2 treatment restored cellular proliferation (FIG. 6C). FIG. 6D-FIG. 6F. β-gal staining of senescent cells. TNFα treatment promoted ß-gal+cell senescence (FIG. 6E) while PFI-2 treatment inhibited TNFα-induced cell senescence (FIG. 6F). FIG. 6G-FIG. 6H. Subcutaneous implantation of collagen sponges coated with PBS (FIG. 6G), TNFα(FIG. 6H) or TNFα plus PFI-2 (FIG. 6I). In this study, TNFα coating increased cell infiltration (FIG. 6H), while PFI-2 reversed this effect (FIG. 6I). In addition, PFI-2 promoted extracellular matrix remodeling (FIG. 61). FIG. 6J-FIG. 6L.

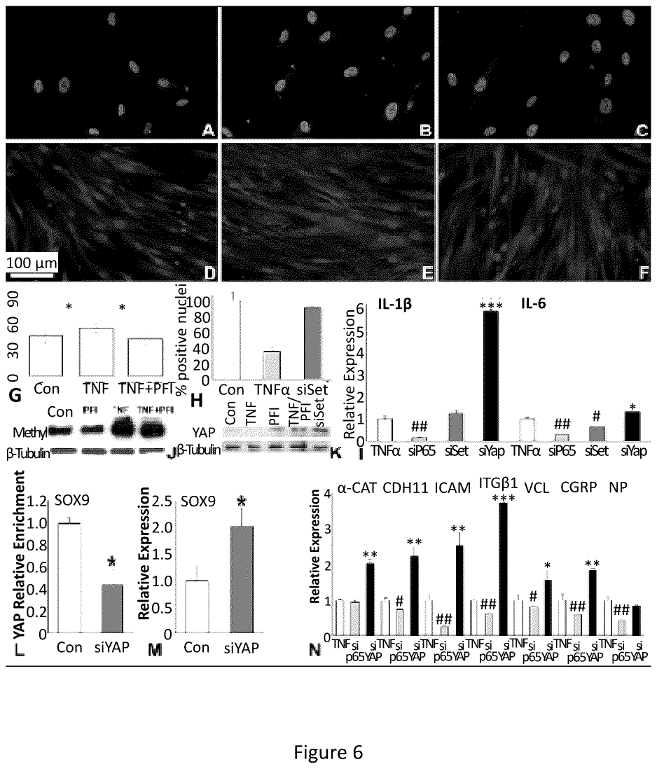

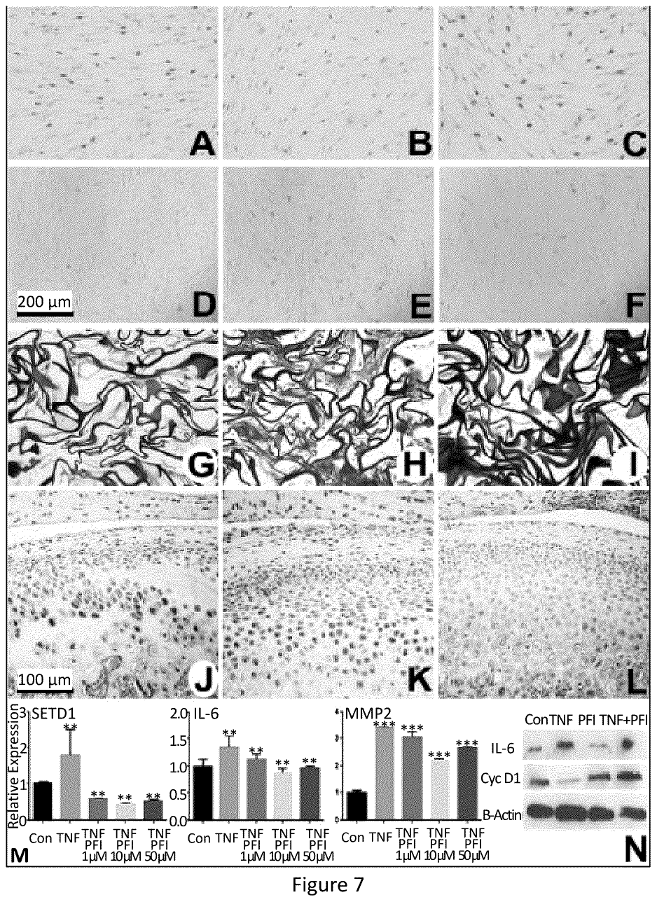

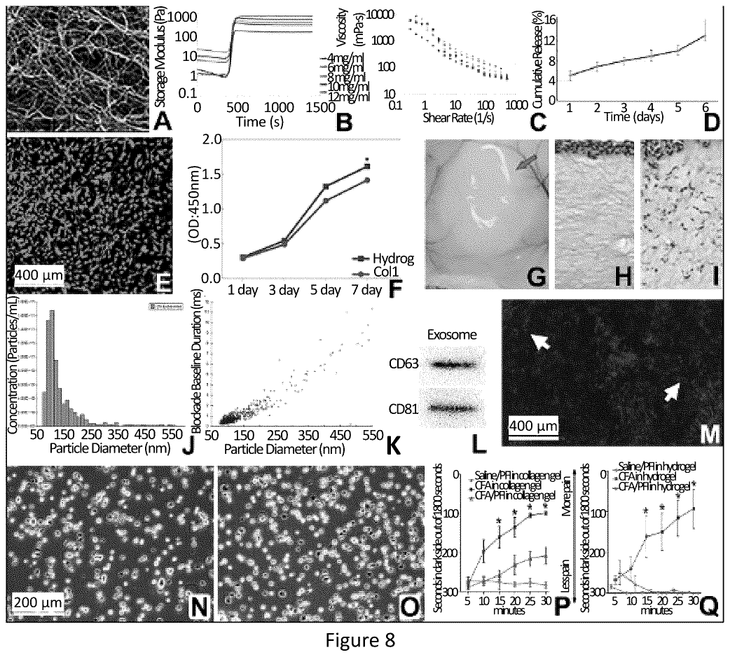

Immunostaining of IL-1β expression in cultured condyle organs treated with BSA (FIG. 6J), IL-6 (FIG. 6K) or IL-6 plus PFI-2 (FIG. 6L). Note that IL-6 treatment increased the thickness of the fibrous and proliferative cell zones and enhanced IL-1β expression in these zones (FIG. 6K). In comparison, PFI-2 treatment reversed the swelling of the articular surface and inhibited IL-1β expression (FIG. 6L). FIG. 6M. qRT-PCR analysis of selected inflammatory genes. TNFα PFI-2 treatment increased SETD1, IL-6 and MMP2 expression, while PFI-2 treatment inhibited TNFα-induced upregulation in a dose-dependent manner (1, 10, and 50 μM). FIG. 6N. Western blotting confirmed the effect of PFI-2 on TNFα-induced gene dysregulation.

FIG. 7, comprising FIG. 7A through FIG. 7Q, depicts representative characterization of PFI-2 carrier materials, dECM hydrogel and RSSC-derived exosomes. FIG. 7A-FIG. 7I. Characterization of the dECM hydrogel. FIG. 7A. SEM image of the dECM hydrogel from decellularized porcine TMJ discs, illustrating its fibrous organization. FIG. 7B-FIG. 7D rheological characterization of the TMJ dECM hydrogels at varying concentrations; FIG. 7B=temperature sweep, FIG. 7C=flow behavior, FIG. 7D=frequency sweeps. FIG. 7E and FIG. 7F. Cell viability analysis using the cell counting kit-8 (Beyotime). The cell proliferation rate was higher when cells were cultured in dECM hydrogel compared to collagen I hydrogel (FIG. 7F). FIG. 7G-FIG. 7I. Subcutaneous injection of dECM hydrogel. One hour after injection the implant was devoid of cells (FIG. 7H) while 7 days after injection, multiple cells had migrated into the implant as revealed by H&E staining (FIG. 7I). FIG. 7J-FIG. 7M. Characterization of exosomes isolated from RSSCs. FIG. 7J and FIG. 7K. Exosome size and distribution. The mean size of the exosomes was 141 nm. FIG. 7L. Western blot analysis of the exosomes demonstrating CD63 and CD81 marker protein expression. FIG. 7M. Fluorescent image of exosome uptake by RSSCs. Fluorescence-labeled exosomes were identified in RSSCs (arrowheads). FIG. 7N and FIG. 70. Cell attachment analysis. Collagen gel (FIG. 7N) or TMJ disc hydrogel (FIG. 70) was used to coat culture dishes for RSSC seeding. Note that more cells were attached on the TMJ disc hydrogel coated surface, indicating that the TMJ-disc hydrogel specifically targets RSSCs. FIG. 7P and FIG. 7Q. PEAP behavior following saline, CFA, or CFA plus PFI-2 injection using collage gel (FIG. 7P) or dECM hydrogel (FIG. 7Q) as carriers. PFI-2 treatment significantly increased the time spent in the dark half of the chamber shortened by CFA injection. This rescue effect was further improved when the dECM hydrogel was used as a carrier (FIG. 7Q).

### DETAILED DESCRIPTION OF THE INVENTION

The present invention relates in part to compositions and methods for treating one or more temporomandibular joint and muscle disorders (TMD) in a subject in need thereof. The present invention is based, in part, on the unexpected discovery that the SET domain containing 7, histone lysine methyltransferase (SETD7) inhibitor PFI-2 is a potent agent for reducing or alleviating pain, particularly inflammatory pain associated with TMD. Accordingly, in some embodiments, the present disclosure provides compositions comprising PFI-2 or a derivative, prodrug, or pharmaceutically acceptable salt thereof for treatment of TMD, as well as methods for reducing or alleviating pain in a subject in need thereof. In one embodiment, the invention comprises a method comprising administering to the subject a therapeutically effective amount of a composition comprising PFI-2 or a derivative, prodrug, or pharmaceutically acceptable salt thereof.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, ]“an element” means one element or more than one element.

“About” as used herein when referring to a measurable value such as an amount, a temporal duration, and the like, is meant to encompass variations of ±20%, ±10%, ±5%, ±1%, or +0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

A “therapeutic” treatment is a treatment administered to a subject who exhibits signs or symptoms of pathology disease or disorder, for the purpose of diminishing or eliminating those signs or symptoms.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a sufficient amount of an agent to provide the desired biological or physiologic result. That result may be reduction and/or alleviation of a sign, a symptom, or a cause of a disease or disorder, or any other desired alteration of a biological system. An appropriate effective amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “potency” refers to the dose needed to produce half the maximal response (ED₅₀).

As used herein, the term “efficacy” refers to the maximal effect (Eₘₐₓ) achieved within an assay.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances, or otherwise evaluating the values or categorization of the substance or the sample.

As used herein, “associated” refers to coincidence with the development or manifestation of a disease, condition, or phenotype. Association may be due to, but is not limited to, genes responsible for housekeeping functions, those that are part of a pathway that is involved in a specific disease, condition, or phenotype and those that indirectly contribute to the manifestation of a disease, condition or phenotype.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

In various embodiments, the present disclosure provides compositions comprising (R)-8-fluoro-N-(1-oxo-1-(pyrrolidine-1-yl)-3-(3-(trifluoromethyl)propan-2-y1)-1,2,3,4-tetrahydroisoquinolone-6-sulfonamide (PFI-2), or a derivative, prodrug, or pharmaceutically acceptable salt thereof.

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers /d/ or diastereomers. Resolution of compounds and isomers thereof is achieved by any means including, by way of non-limiting example, chemical processes, enzymatic processes, fractional crystallization, distillation, and chromatography.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically, or therapeutically active form of the compound. In another embodiment, a prodrug is enzymatically metabolized by one or more steps or processes to the biologically, pharmaceutically, or therapeutically active form of the compound.

In some embodiments, the composition is formulated for sustained and/or controlled-release of PFI-2. In some embodiments, the composition comprises a gel, a hydrogel, an exosome, a nanoparticle, an exosome, or a combination thereof.

In some embodiments, the composition comprises PFI-2, or a derivative, prodrug, or pharmaceutically acceptable salt thereof, dissolved in, suspended in, or embedded in a hydrogel.

In some embodiments, the composition comprises one or more hydrogels. Hydrogels can generally absorb a great deal of fluid and, at equilibrium, typically are composed of 60-90% fluid and only 10-30% polymer. Hydrogels are particularly useful due to the inherent biocompatibility of the cross-linked polymeric network (Hill-West, et al., 1994, Proc. Natl. Acad. Sci. USA 91:5967-5971). Hydrogel biocompatibility may be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27).

Hydrogels closely resemble the natural living extracellular matrix (Ratner and Hoffman. Synthetic Hydrogels for Biomedical Applications in Hydrogels for Medical and Related Applications, Andrade, Ed. 1976, American Chemical Society: Washington, D.C., pp 1-36). Hydrogels may also be made degradable in vivo by incorporating PLA, PLGA or PGA polymers. Moreover, hydrogels may be modified with fibronectin, laminin, vitronectin, or, for example, RGD for surface modification, which may promote cell adhesion and proliferation (Heungsoo Shin, 2003, Biomaterials 24:4353-4364; Hwang et al., 2006 Tissue Eng. 12:2695-706). Indeed, altering molecular weights, block structures, degradable linkages, and cross-linking modes may influence strength, elasticity, and degradation properties of the instant hydrogels (Nguyen and West, 2002, Biomaterials 23(22): 4307-14; Ifkovits and Burdick, 2007, Tissue Eng. 13(10): 2369-85).

The hydrogels may be prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin, agarose, alginates, cellulose, starch, and extracellular matrix (EC). These materials consist of high-molecular weight backbone chains made of linear or branched polysaccharides, polypeptides, or combinations thereof. Examples of hydrogels based on chemical or physical crosslinking synthetic polymers include but are not limited to (meth)acrylate-oligolactide-PEG-oligolactide-(meth)acrylate, poly(ethylene glycol) diacrylate (PEGDA), poly(ethylene glycol) (PEG), poly(propylene glycol) (PPG), poly(lactic acid) (PLA), poly(lactic-co-glycolic acid) (PLGA), poly(caprolactone) (PCL), poly(phosphazene), poly(methacrylates), poly(N-vinylpyrrolidone), poly(ethylene imine) (PEI), and copolymers and combinations thereof. (see A. S Hoffman, 2002, Adv. Drug Del. Rev, 43, 3-12).

The hydrogel can be cross-linked based on the type(s) of hydrogel used, such as by photo-cross-linking, thermal-cross-linking, chemical cross-linking, and the like. Hydrogels may also be modified with functional groups for covalently attaching a variety of proteins or compounds such as therapeutic agents. It is contemplated that linkage of the therapeutic agent to the hydrogel may be via a protease sensitive linker or other biodegradable linkage.

In some embodiments, the hydrogel comprises at least one biopolymer. In some embodiments, the biopolymer is one or more selected from the group consisting of fibrinogen, collagen, hyaluronic acid, alginate, and ECM.

In some embodiments, the hydrogel is injectable. In some embodiments, the hydrogel is a collagen hydrogel. In some embodiments, the collagen hydrogel is a collagen type I (Col I) hydrogel. In some embodiments, the collagen hydrogel comprises collagen in a concentration between about 1 mg/mL and about 50 mg/mL. In some embodiments, the collagen concentration is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 25 mg/mL, about 30 mg/mL, about 35 mg/mL, about 40 mg/mL, about 45 mg/mL, or about 50 mg/mL. In some embodiments, the collagen hydrogel has a pH between about 6 and about 8. In some embodiments, the collagen hydrogel has a pH of 7. In some embodiments, the collagen hydrogel comprises PFI-2, or a derivative, prodrug, or pharmaceutically acceptable salt thereof, at a concentration between about 100 nM and about 100 mM. In some embodiments, the concentration is about 100 nm, about 200 nM, about 300 nM, about 400 nM, about 500 nM, about 600 nM, about 700 nM, about 800 nM, about 900 nM, about 1μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 25 μM, about 30 μM, about 35 μM, about 40 μM, about 45 μM, about 50 μM, about 60 μM, about 70 μM, about 80 μM, about 90 μM, about 100 μM, about 200 μM, about 300μM, about 400 μM, about 500 μM, about 600 μM, about 700 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mm, about 20 mM, about 25 mM, about 30 mM, about 35 mM, about 40 mM, about 45 mM, about 50 mM, about 60 mM, about 70 mM, about 80 mM, about 90 mM, or about 100 mM.

In some embodiments, the hydrogel is an ECM hydrogel. In some embodiments, the ECM is derived from TMJ tissue or the surrounding tissue. In some embodiments, the ECM is derived from TMJ disc tissue. In some embodiments, the ECM hydrogel is a decellularized ECM (dECM) hydrogel. In some embodiments, the dECM is produced by decellularizing ECM with a protease, a nuclease, and a detergent. In some embodiments, the protease is trypsin. In some embodiments, the detergent is Triton® X-100.

In some embodiments, the dECM hydrogel comprises dECM in a concentration between about 1 mg/mL and about 50 mg/mL. In some embodiments, the collagen concentration is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 25 mg/mL, about 30 mg/mL, about 35 mg/mL, about 40 mg/mL, about 45 mg/mL, or about 50 mg/mL. In some embodiments, the dECM hydrogel has a pH between about 6 and about 8. In some embodiments, the dECM hydrogel has a pH of 7. In some embodiments, the collagen hydrogel comprises PFI-2, or a derivative, prodrug, or pharmaceutically acceptable salt thereof, at a concentration between about 100 nM and about 100 mM. In some embodiments, the concentration is about 100 nm, about 200 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 25 μM, about 30 μM, about 35 μM, about 40 μM, about 45 μM, about 50 μM, about 60 μM, about 70 μM, about 80 μM, about 90 μM, about 100 μM, about 200 μM, about 300 μM, about 400 μM, about 500μM, about 600 μM, about 700 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mm, about 20 mM, about 25 mM, about 30 mM, about 35 mM, about 40 mM, about 45 mM, about 50 mM, about 60 mM, about 70 mM, about 80 mM, about 90 mM, or about 100 mM.

In some embodiments, compositions of the present invention comprise PFI-2, or a derivative, prodrug, or pharmaceutically acceptable salt thereof, suspended in or encapsulated in an exosome. In some embodiments, the exosomes are derived from mesenchymal stem cells (MSCs), embryonic stem cells (ESCs), induced-pluripotent stem cells (iPSCs), or a combination thereof. In some embodiments, the exosomes are derived from synovial MSCs. In some embodiments, the exosomes comprise PFI-2, or a derivative, prodrug, or pharmaceutically acceptable salt thereof, at a concentration between about 100 nM and about 100 mM. In some embodiments, the concentration is about 100 nm, about 200 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 25 μM, about 30 μM, about 35 μM, about 40 μM, about 45 μM, about 50 μM, about 60 μM, about 70 μM, about 80 μM, about 90μM, about 100 μM, about 200 μM, about 300 μM, about 400 μM, about 500 μM, about 600 μM, about 700 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mm, about 20 mM, about 25 mM, about 30 mM, about 35 mM, about 40 mM, about 45 mM, about 50 mM, about 60 mM, about 70 mM, about 80 mM, about 90 mM, or about 100 mM.

In some embodiments, compositions of the present invention comprise PFI-2, or a derivative, prodrug, or pharmaceutically acceptable salt thereof, suspended in or encapsulated by a nanoparticle. In some embodiments, the nanoparticles comprise one or more selected from the group consisting of collagen, ECM, and polyethylene glycol (PEG). In some embodiments, the nanoparticles comprise between about 0.0001% and about 50% PFI-2, or a derivative, prodrug, or pharmaceutically acceptable salt thereof, by weight. In some embodiments, the nanoparticles comprise about 0.0001%, about 0.0005%, about 0.001%, about 0.005%, about 0.01%, about 0.05%, about 0.1%, about 0.5%, about 1%, about 5%, about 10%, or about 50% PFI-2 by weight.

In some embodiments, compositions of the present invention comprise one or more hydrogels, exosomes, or nanoparticles of the present invention suspended in a carrier. In some embodiments, the carrier is one or more selected from the group consisting of water, saline, and phosphate-buffered saline (PBS). In some embodiments, the concentration of the hydrogel, exosome, or nanoparticle in the composition is between about 100 ng/ml and about 100 mg/mL. In some embodiments, the concentration is about 100 ng/mL, about 200 ng/ml, about 300 ng/ml, about 400 ng/ml, about 500 ng/mL, about 600 ng/ml, about 700 ng/ml, about 800 ng/ml, about 900 ng/ml, about 1 μg/mL, about 2 g/mL, about 3 μg/mL, about 4 μg/mL, about 5 μg/mL, about 6 μg/mL, about 7 μg/mL, about 8 μg/mL, about 9 μg/mL, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 60 μg/mL, about 70 μg/mL, about 80 μg/mL, about 90 μg/mL, about 100 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1 mg/mL, about 2 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about or about 100 mg/mL. In some embodiments, the average concentration of PFI-2, or a derivative, prodrug, or pharmaceutically acceptable salt thereof, in the carrier is between about 100 nM and about 100 mM. In some embodiments, the concentration is about 100 nm, about 200 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 25 μM, about 30 μM, about 35 μM, about 40 μM, about 45 μM, about 50 μM, about 60 μM, about 70 μM, about 80 μM, about 90 μM, about 100 μM, about 200 μM, about 300 μM, about 400 μM, about 500 μM, about 600 μM, about 700 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mm, about 20 mM, about 25 mM, about 30 mM, about 35 mM, about 40 mM, about 45 mM, about 50 mM, about 60 mM, about 70 mM, about 80 mm, about 90 mm, or about 100 mm.

In another aspect, the present invention relates to the unexpected discovery that decreased levels of YAP increase inflammation and inflammatory pain.

For example, decreased levels of YAP in TMJ tissue results in TMDs. As YAP degradation occurs when the Hippo pathway is active, in some embodiments, compositions of the present invention comprise one or more inhibitors of the Hippo pathway. In some embodiments, a composition of the invention comprises a composition comprising PFI-2 of the present invention further comprising an inhibitor of the Hippo pathway.

### Methods of Treatment

In various embodiments, the disclosure provides methods of reducing or alleviating pain. In some embodiments the pain is an inflammatory pain. In some embodiments, the method comprises administering a therapeutically effective amount of a composition of the present invention to a subject in need thereof. In some embodiments, the method comprises administering the composition by injection.

In some embodiments, the inflammatory pain is associated with the temporo-mandibular joint (TMJ). In some embodiments, the inflammatory pain is associated with a TMJ disorder (TMD). Examples of TMDs include, but are not limited to, synovitis, capsulitis, retrodiscitis, ligamentitis, and arthritis of the TMJ. Accordingly, in some embodiments, the present disclosure provides methods of treatment for TMD.

In some embodiments, the method comprises administering a therapeutically effective amount of a composition of the present invention to a subject in need thereof. In certain embodiments, the subject has inflammatory pain associated with the TMJ. For example, in one embodiment, the subject has a TMD. In some embodiments, the method comprises administering the composition by injection. In some embodiments, the composition is injected directly into the TMJ and/or the surrounding tissue.

### Methods of Screening

In another aspect, the present disclosure relates to a model for TMDs, a masseter tendon ligament model. Accordingly, in some embodiments, the present disclosure provides methods of screening for compounds useful for the treatment of TMDs utilizing the TMD model. In some embodiments, the method comprises the step of administering a compound of interest (test) and a control to an animal with pain hypersensitivity in the masseter muscle and comparing an increase or decrease in pain response in the test animal relative to the control animal.

### Pharmaceutical Compositions and Formulations

The invention also encompasses the use of pharmaceutical compositions to practice the methods of the invention. Such pharmaceutical compositions may comprise at least one compound of the invention or a salt thereof in a form suitable for administration to a subject, or the pharmaceutical compositions may comprise at least one compound of the invention or a salt thereof, and one or more pharmaceutically acceptable carriers, one or more additional ingredients, or some combination of these. The compound or conjugate thereof may be present in the pharmaceutical composition in the form of a physiologically acceptable salt, such as in combination with a physiologically acceptable cation or anion, as is well known in the art.

Pharmaceutical compositions that are useful in the methods of the invention may be suitably developed for oral, rectal, vaginal, parenteral, topical, pulmonary, intranasal, buccal, ophthalmic, or another route of administration. The pharmaceutical compositions useful within the methods of the invention may be directly administered to the skin, vagina, or any other tissue of a mammal. Other contemplated formulations include liposomal preparations, resealed erythrocytes containing the active ingredient, and immunologically based formulations. The route(s) of administration will be readily apparent to the skilled artisan and will depend upon any number of factors including the type and severity of the disease being treated, the type and age of the veterinary or human subject being treated, and the like.

Although the invention herein is principally directed to the ethical administration of the pharmaceutical compositions to humans, it will be understood by the skilled artisan that such compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist may design and perform such modification with merely ordinary, if any, experimentation. Subjects to which administration of the pharmaceutical compositions is contemplated include, but are not limited to, humans and other primates, mammals including commercially relevant mammals such as cattle, pigs, horses, sheep, cats, and dogs.

In one embodiment, the pharmaceutical compositions utilized in the invention are formulated using one or more pharmaceutically acceptable excipients or carriers. In one embodiment, the pharmaceutical compositions comprise a therapeutically effective amount of a compound or conjugate thereof of the invention and a pharmaceutically acceptable carrier. Pharmaceutically acceptable carriers that are useful, include, but are not limited to, glycerol, water, saline, ethanol, and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In one embodiment isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical compositions may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition utilized in the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the pharmaceutical composition includes an antioxidant and a chelating agent that inhibits the degradation of the compound. Exemplary antioxidants for some compounds are BHT, BHA, alpha-tocopherol, and ascorbic acid in the range of about 0.01% to 0.3%. In one embodiment, the BHT is in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g., disodium edetate) and citric acid in the weight range of about 0.01% to 0.20%. In one embodiment, chelating agents may be in the range of 0.02% to 0.10% by weight by total weight of the composition. The chelating agent is useful for chelating metal ions in the composition that may be detrimental to the shelf life of the formulation. While BHT and disodium edetate are the exemplary antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition for use in the invention may comprise each of the components described regarding liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the composition utilized in the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition for use in the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the pharmaceutical compositions of the present invention to a subject, such a mammal, including a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily, or the dose may be proportionally reduced as indicated by the exigencies of the therapeutic situation. A non-limiting example of an effective dose range for a therapeutic compound for use in the invention is from about 1 and 5,000 mg/kg of body weight/per day. One of ordinary skill in the art would be able to study the relevant factors and make the determination regarding the effective amount of the therapeutic compound without undue experimentation.

The invention may be practiced as frequently as several times daily, or it may be practiced less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In certain embodiments, the pharmaceutical composition of the present invention provides for a controlled release of a therapeutic agent. In certain instances, controlled- or sustained-release formulations of a pharmaceutical composition of the invention may be made using conventional technology, using for example proteins equipped with pH sensitive domains or protease-cleavable fragments. In some cases, the dosage forms to be used can be provided as slow or controlled release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, lozenges, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased subject compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In certain embodiments, the controlled-release formulation of the composition described herein allows for release of a therapeutic agent precisely when the agent is most needed. In another embodiment, the controlled-release formulation of the composition described herein allows for release of a therapeutic agent precisely in conditions in which the therapeutic agent is most active. In order to maintain this constant level of drug in the body, the drug must be released from the dosage form at a rate that will replace the amount of drug being metabolized and excreted from the body.

In certain embodiments, the pharmaceutical composition provides for an environment-dependent release, when and where the therapeutic agent is triggered for release. For example, in certain embodiments the composition invention releases at least one therapeutic agent when and where the at least one therapeutic agent is needed. The triggering of release may be accomplished by a variety of factors within the microenvironment of the treatment or prevention site, including, but not limited to, temperature, pH, the presence or activity of a specific molecule or biomolecule, and the like.

Controlled release of an active ingredient can be stimulated by various inducers, for example pH, temperature, enzymes, water or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid offset, as well as controlled, for example, sustained release, delayed release, and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the pharmaceutical compositions are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

In one embodiment, the invention is practiced in dosages that range from one to five times per day or more. In another embodiment, the invention is practiced in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Routes of administration of include oral, nasal, rectal, parenteral, sublingual, transdermal, transmucosal (e.g., sublingual, lingual, (trans)buccal, (trans)urethral, vaginal (e.g., trans-and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions, and formulations for intravesical administration and the like. It should be understood that the formulations and compositions that would be useful in the present invention are not limited to the particular formulations and compositions that are described herein.

### EXPERIMENTAL EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Without further description, it is believed that one of ordinary skill in the art can, using the preceding description and the following illustrative examples, make and utilize the present invention and practice the claimed methods. The following working examples therefore, point out specific embodiments of the present invention, and are not to be construed as limiting in any way the remainder of the disclosure.

### Example 1

### Characterization of the Involvement of PFI-2 in TMD Inflammation-Induced and YAP-Associated TMD Degeneration and Pain

The studies described herein determined the effect of a SetD7 histone demethylase inhibitor, the small molecule probe PFI-2, on inflammatory pain. This inhibitor was tested in an inflammatory temporo-mandibular joint disorder model (TMD) that was generated by injecting Freud's adjuvant into the joint area. In these studies, application of the small molecules to mice subjected to inflammatory temporo-mandibular joint pain spent a dramatically reduced time on the dark side of place escape/avoidance paradigm (PEAP) chambers, indicative of greatly reduced pain. These PEAP chamber measure the aversion to mechanically evoked pain.

“Despite investment in research directly and indirectly related to temporomandibular disorders (TMD)-most significantly in the field of orofacial pain researchers have yet to unravel the etiologies and pathophysiologies of TMD”, states a current consensus report by the National Academies of Sciences (Bond et al. 2020). To address this knowledge gap, the present studies have focused on the pain associated with inflammatory TMDs including osteoarthritic and myofascial components. In preliminary studies, a significant 30% reduction of YAP (yes associated protein 1) was detected associated with an inflammatory TMD model and significantly increased levels of the YAP monomethyltransferase SETD7 (1.6-fold), pointing to a role of the mechanotransduction associated Hippo pathway and its key transcriptional effectors YAP/TAZ as candidate contributors toward inflammatory TMD pathogenesis. This investigative lead was further confirmed by the effectiveness of a small molecule inhibitor, PFI-2, in modulating acute TMD pain.

The present disclosure demonstrates a highly innovative and radical departure from current approaches toward the understanding of temporomandibular joint disorders and treatment in at least three aspects. The first novel aspect is that this is the first study implicating the Hippo pathway in temporomandibular joint function and dysfunction. Use of Yap mutant mice to study changes in TMJ function and TMD disease progression as a result of Hippo pathway alteration is also entirely new. The second novel aspect is the highly innovative and paradigm-shifting the use of PFI-2 as a therapeutic aid to interfere with SETD7-YAP activity and restore the tissue damage and pain associated with inflammatory disease. The therapeutic application of PFI-2 is novel and so is its use in the treatment of TMD. Finally, the use of the masseter tendon ligament model as an alternative to the traditional CFA model for the study of chronic TMJ inflammation and pain is unique and novel.

Temporomandibular joint disorders (TMDs) are disorders of complex etiology often characterized by debilitating TMJ or myofascial muscle pain and/or limitations in mouth opening (Bond et al. 2020). Inflammatory joint disease begins with inflammatory cell infiltration and inflammation-related gene dysfunction, which then causes temporomandibular joint degeneration, characterized by a breakdown of the articular cartilage, changes in bone architecture, and a destruction of the synovial tissues (Wang et al. 2012, Slade et al. 2011). Key inflammatory mediators include IL-1β, IL-6. L-8, and TNF-α, which lead to tissue degeneration through the regulation of metalloproteinase expression. Inflammatory pain is characterized by the interactions of inflammatory mediators with neurons to produce hypersensitivity and involves a series of receptors, ion channels and neural transmitters and neuropeptides (Kidd and Urban, 2001). Neuropeptides such as substance P (SP), neuropeptide Y (NPY) and calcitonin gene-related peptide (CGRP) are also expressed by synovial tissues under inflammatory conditions (Wang et al. 2015; Liu et al. 2014).

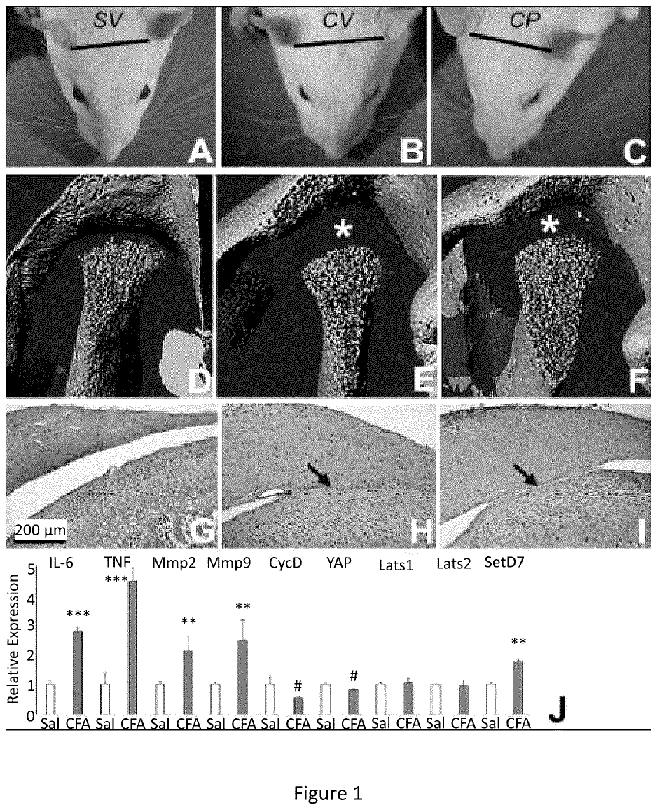

As a preliminary study, rats were injected with saline, complete Freund's adjuvant (CFA), or CFA and PFI-2 (FIG. 1). After 48 hours, regional swelling was observed in the CFA rat, with a 10% increase in distance between the two ear tragi, while no swelling was observed in rats injected with CFA and PFI-2. To further verify the observed swelling, uCT images were taken of the condyles and temporal fossae of the rats, where a visible decrease in condylar bone volume and increase in joint space was visible for rats injected with CFA, which was rescued by addition of PFI-2.

The tissue was further investigated by H&E staining TMJ tissue embedded in paraffin, which revealed thickening of the TMJ disc and fusion with the condyle. An increase in thickness of the fibrous and proliferative cell zones was observed, while a decrease in thickness was observed in the pre-hypertrophic and hypertrophic zones, all of which were rescued by treatment with PFI-2. qRT-PCR analysis of inflammatory genes in the tissue, and YAP pathway members revealed a changes not only in neuropeptide signaling, but also resulted in a significant 30% reduction in YAP and a 1.6-fold increase in the YAP monomethyltransferase SETD7 (FIG. 1), prompting investigations into whether the YAP pathway was involved in inflammatory TMD, changes in neuropeptide expression, and pain.

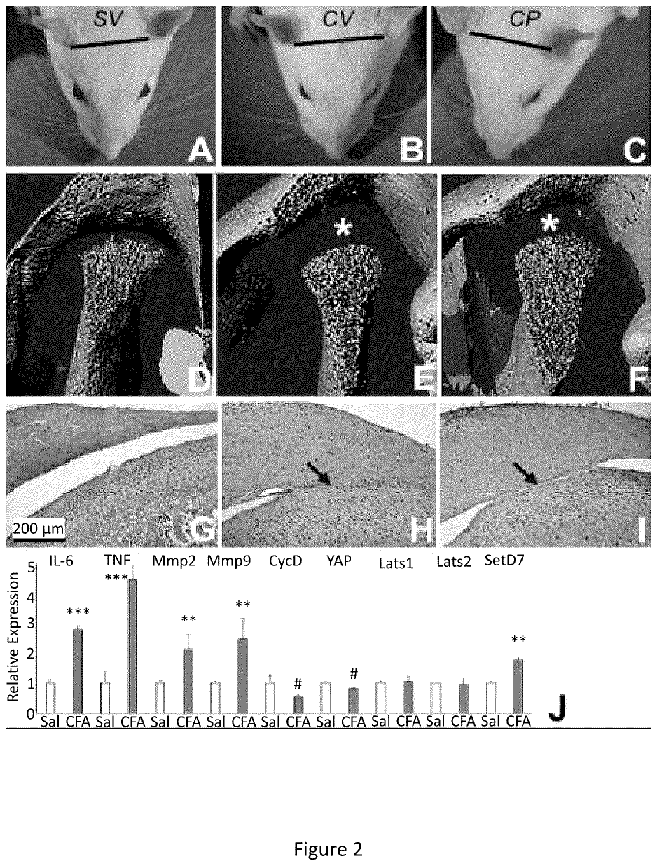

In light of this, and studies that have demonstrated that the small molecule PFI-2, a specific inhibitor of the methyltransferase SETD7, suppresses inflammation and promotes tissue regeneration in periodontal diseases (Francis et al. 2018), additional investigations of the effects of PFI-2 treatment on inflammation-induced degeneration of the TMJ matrix, and YAP1 expression, were performed. Masson trichrome images of the tissue sections depicted in FIG. 1G-FIG. 1I revealed fibrous adhesion of TMJ disc and condylar surface following CFA-induced inflammation, while PFI-2 treatment prevented the attachment (FIG. 2A-FIG. 2C). Pico Sirius red staining revealed yellow-green thin collagen fibers in the control group, compared to dark red, thick fibers in the CFA-treated tissue, which was lightened in samples from PFI-2 treated animals (FIG. 2D-FIG. 2F).

YAP-1 immuno-localization in TMJ discs demonstrated an increase in YAPI expression in CFA-treated animals compared to controls, while PFI-2 treatment translocated YAP into nuclei (FIG. 2G-FIG. 2I). qRT-PCR analysis of the samples for matrix protein and adhesion molecule expression revealed an upregulation in the expression of matrix proteins and adhesion molecules, except collagen 2) in animals treated with CFA, which was reversed in animals treated with PFI-2.

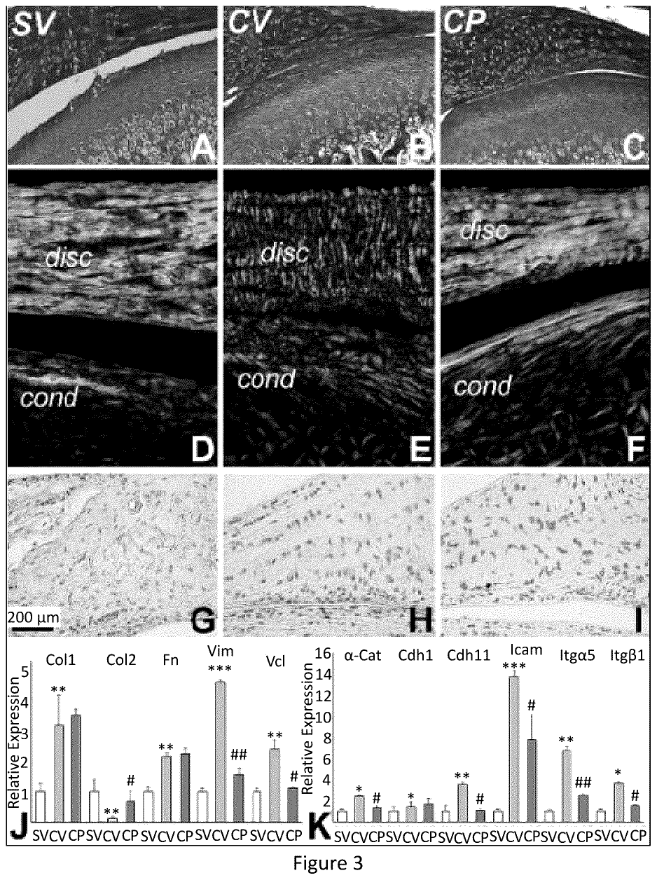

Because of this initial success, it was further examined if PFI-2 treatment could rescue inhibition of chondrocyte maturation and return YAP expression in the condylar growth plate of animals suffering from inflammation. To test the ability to repair damage, animals were injected with saline or CFA. After inflammation, a subset of the CFA-treated animals were also injected with PFI-2. Tissue samples from each were stained with H&E, safranin O, and PCNA, YAP was detected by immunostaining, and cartilage differentiation was determined by immunostaining for Sox 9 (FIG. 3A-FIG. 3O). The samples were also examined by RT-PCR for expression levels of collagen 2, collagen 10, Mmp 13 metalloproteinase, Runx2 bone transcription factor, and Sox 9 (FIG. 3P). Collectively, the results demonstrate that PFI-2 treatment is not only able to prevent inflammation and inflammation-induced damage, but able to reverse it and rebalance YAP1 expression.

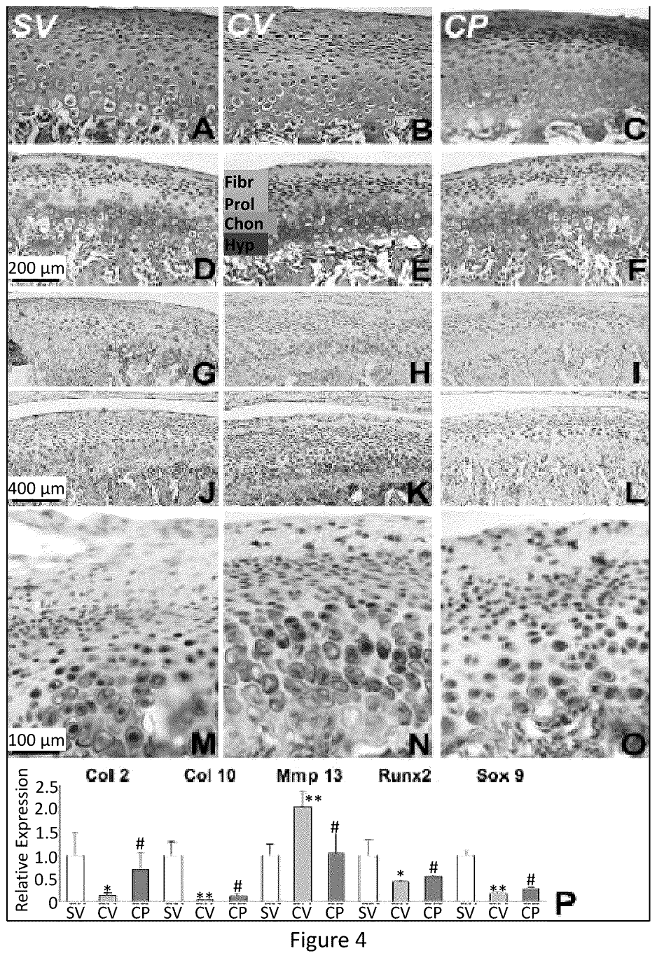

To see if this reduction in inflammation and its associated damage could provide therapeutic benefit, the ability of PFI-2 to reduce inflammatory pain was examined. Animals were injected with saline or CFA and meal duration, a proxy for pain, was examined over a span of 21 days post-injection. A significant and constant increase in meal duration was observed in animals that had been injected with CFA (FIG. 4A and FIG. 4B). Calcitonin gene-related peptide (CGRP) localization and expression in the synovial membrane were examined by immunostaining, where it was observed that CGRP was localization in synovial cells and neurons was increased in animals treated with CFA (FIG. 4C and FIG. 4D). Given the altered neuropeptide expression and localization, additional neuropeptides and their receptors were examined by qRT-PCR in animals treated with saline, CFA, or CFA and PFI-2, where an increase in CGRP, NPY, and TAC was observed in CFA-treated animals, which was reduced to near-normal levels with PFI-treatment, demonstrating a reduction in inflammatory pain with PFI-2 treatment (FIG. 4E)).

Assessment of Inflammation-induced Tissue Changes in TMDs: for These studies a CFA injection rat model, described above (FIG. 1), is used. Twenty-four hours following the first CFA injection, external signs of TMD including swelling and chromodacryorrhea are graded every day. After 12 days, the TMJ discs, synovia and condyles are collected bilaterally for histology, RNA and protein isolation. Subchondral bone degeneration are evaluated using a Scanco 40 μCT apparatus (Scanco Medical, Wayne, PA). Trabecular bone volume/total volume (BV/TV), trabecular thickness (Tb/Th), trabecular separation (Tb/Sp), and trabecular number (TbN) are calculated. Separate cortical bone thickness (Cr/Th) and bone mineral density (BMD) will be measured as previously described by Lu at al. (2013 and 2016a, b). For histology, the discs, synovium and condyles are removed, fixed, and demineralized. Approximately 40 paraffin sections of 5 μm thickness are cut sagittally through the mid-condylar region and subjected to the following staining protocols: (i) H&E staining to measure tissue thickness and cell numbers, (ii) Toluidine Blue and Safranin O staining to determine proteoglycan changes, (iii) Masson trichrome staining to characterize changes in trabecular bone morphology and non-mineralized bone matrix, and (iv) TRAP staining to assess osteoclast activity as described by Lu et al. (2013). In addition, the three centermost sections on each side are subjected to morphometric measurements to determine disc thickness, condylar cartilage thickness, and thickness of the proteoglycan-rich region for the anterior band, intermediate zone, and posterior band as described by Wang et al. (2012). In addition, the total number of mononucleated cells in each region is counted and averaged per region.

Expression of Osteogenic, Chondrogenic and Inflammatory Genes in a TMD model: To generate benchmark data for functional and mechanistic studies TMJ discs, synovia, and condyles are dissected and mRNA and protein expression levels of bone marker genes measured, including collagen type I (Col I), alkaline phosphatase (Alp), Runx2, Osterix (Osx), osteocalcin (Ocn), and bone sialoprotein (Ibsp), as well as cartilage marker genes such as collagen II, Sox 9, Col X, and biglycans. To characterize the effect of the YAP upregulator PFI-2 on inflammation and osteoclast formation, the expression of inflammatory cytokines (IL-1b, IL-6, TNF-α), NF-κB, MMPs (MMP2, MMP9 and MMP13), adhesion molecules (CDH11, ICAM, Integrin β1), and osteoclastogenesis-related genes (Rank, Rankl, Opg, Trap5b, and NFATc1) is detected using quantitative RT-PCR and Western blotting as described by Walker et al. (2010). Localization of these genes in the inflamed TMJ is further specified using immunofluorescence.

Effect of PFI-2 Mediation on Inflammatory Conditions and Pain: Tmj pain is assessed as meal duration and via Place Escape Avoidance Paradigm (PEAP)-based measures (FIG. 1, Vertebrate Justification section). Testing is performed in both male and female rats as sex differences have been observed in humans and animals. Female animals are tested for the estrous cycle and the results are reported for the di-estrus, estrus, and pro-estrus phases (Kramer, Bellinger, 2009).

Meal Duration and Feeding Behavior Studies. the Lengthening of Meal duration during TMJ pain is a “guarding behavior”, an operationally defined nociceptive behavior (Sternberg and Wachterman, 2000). To examine changes in meal duration, rats are housed individually in sound-attenuated chambers equipped with photobeam computer-activated pellet feeders (Med Assoc. Inc., East Fairfield, VT, USA). A meal is defined using a 10-minute end-of-meal criterion, and the minimum meal size is set at 135 mg (Castonguay et al., 1986). Meal duration is then calculated using proprietary and Med Associate computer programs as previously established (Kerins et al., 2003, 2004; Kramer et al., 2010).

Peap Testing: the Chamber Used for the Place Escape Avoidance Paradigm (PEAP) testing is a 30 cm×30 cm acrylic box in which half the box is covered in black cloth. Each individual rat is placed into the chamber; due to their nocturnal circadian rhythm, rodents prefer the dark side of the chamber. Rats on the dark side of the chamber receive a stimulus at the injected side, whereas rats with their head on the clear side of the chamber are stimulated on the contralateral non-injected side with a 60 g filament every 15 seconds. The stimulus is administered below the eye and caudal to the whisker pad. The time spent on the dark side of the box is recorded in 5 min bins, testing is performed for a total of 30 min on the test day and thereafter on a one day a week basis for 8 weeks as previously described (Stinson et al. 2017; 2019).

Neuronal activity determination: Neuronal activity in peripheral and central parts of the nervous system is determined by focusing on the trigeminal ganglia and the nucleus caudalis of the spinal trigeminal nucleus. Trigeminal ganglia and spinal cord from the spinomedullary junction transition zone containing the trigeminal nucleus caudalis are removed from rats subjected to our inflammatory pain models. Tissues are fixed in 4% paraformaldehyde overnight followed by incubation with 15% sucrose for 1 hour and 30% sucrose overnight at 4° C. Trigeminal ganglia and spinal cord tissues are sagittally cut. Since elevated levels of the phosphorylated forms of the MAP kinases P38 and ERK are associated with peripheral sensitization, while phosphorylation of c-Fos is associated with the activation level of second order sensory neurons within the spinal medullary horn containing the trigeminal nucleus caudalis (Harris, 1998; Jin et al, 2003), the localization and expression of p-P38 and p-ERK (Cell Signaling), and c-Fos (Abcam) is detected using immunofluorescence staining with DAPI as a counterstain. C-Fos immunoreactive cells in the trigeminal nucleus caudalis, as well as p-P38 and p-ERK positive cells in trigeminal ganglia are counted.

Inflammatory Changes in the Expression of Neuropeptides CGRP, NPY and SP1 in vivo and in vitro: For in vivo studies, rat TMJ discs, synovial membrane, condyle, trigeminal ganglia and trigeminal nucleus caudalis are collected bilaterally from saline-or CFA-treated rats for RNA and protein isolation, and histological analysis. The expression of CGRP, NPY, TAC1, and Galanin (GAL) at gene and protein levels are measured using qRT-PCR and Western blotting. For histology, tissues are fixed in 4% paraformaldehyde and prepared for paraffin sections as described previously (Lu et al, 2016a, b). Cells and neurons positive for CGRP, NPY, SP1 (an isoform of TAC1 protein) and Gal are visualized via immunofluorescence or immunohistochemistry using antibodies against CGRP, GAL, NYP, and TAC1 (Abcam). Expression levels of these genes and the numbers of immunoreactive cells are compared between different treatment groups.

For in vitro studies (FIG. 2 and FIG. 3), the synovial mesenchymal stem cells (RSSCs) from the TMJ is used since they are neurogenic and exhibit similar cytological features as neuronal cells (Cady et al. 2011; Liu et al. 2009; Liu et al. 2011). A primary SSC rat cell line (RSSC) has been established (FIG. 2). In the present study, these RSSCs and a dorsal root ganglion neuron cell line, ND7/23 (Sigma), are used for in vitro studies of neuropeptides and their receptor expression. To mimic chronic inflammatory conditions, RSSC progenitor cells are first cultured in the presence of IL-6 (10 ng/ml) or TNF-α (10 ng/ml) for 7 days. To determine the effect of YAP upregulation via SETD7 monomethylation inhibition, the small molecule mediator PFI-2 is administered at concentrations of 1 μM and 10 μM for 3 days and its effect on expression of neuropeptide and receptor expression, including CGRP, CLRL, NYP1, NYP1R, TAC1, TACR1, Gal and GALR1, is assessed.

Determination of Changes in Hippo-yap Signaling Resulting from Inflammatory TMD: The results above have demonstrated degeneration and altered TMJ tissue homeostasis as a result of inflammatory conditions, suggestive of the involvement of Hippo pathway-Yap/TAZ signaling in inflammatory TMDs (FIG. 1). Here, the role of the Hippo pathway effectors YAP and TAZ is elucidated in an inflammatory TMD models. YAP and TAZ interact with DNA-binding factors of the TEAD family in the nucleus to regulate gene expression. Since YAP and TAZ have similar localization in TMJ tissues and the expression level of TAZ was not changed significantly, the main focus of the present disclosure is on the function of YAP in TMDs. First, the expression and subcellular localization of YAP is characterized under physiological and inflammatory conditions in TMJ tissues, RSSCs and RCs. The expression pattern of the YAP protein in TMJ tissues are elucidated by immunohistochemistry using anti-YAP antibody and the expression levels of YAP mRNA and protein is determined using qRT-PCR and Western blotting. To identify possible upstream and downstream genes involved in YAP signaling, RNA-sequencing and gene-set enrichment analyses are performed in disc, synovial membrane, and condylar tissues, and the data verified by RT-PCR. RNAseq and gene-set enrichment studies result in a comprehensive characterization of the inflammation-induced changes in the YAP protein modification enzymes (Lats1 and 2, SETD7) and YAP target genes including cell cycle (Cyclin D1, E1, PCNA), cell growth (OOT4, SOX2, EGFR), and integrin signaling (CYR61, CTGF) regulators that are likely to play a role in inflammatory TMDs. Together, combining the outcomes of gene expression changes of inflammatory regulators and inflammatory pain mediators with trends in YAP specific signaling molecules provides a first assessment of the regulatory involvement of YAP signaling in inflammatory TMDs. These data are integrated with the outcomes of YAP upregulation studies using the SETD7 monomethylation inhibitor PFI-2.

### Example 2

### Mechanistic Determination of PFI-2 Modulation of Hippo Pathway Dysregulation Through SETD7

The Salvador-Warts-Hippo pathway (Hippo pathway) is a key signaling pathway responsible for the control of organ size and the maintenance of tissue homeostasis (Pan 2010, Kim and Jho 2018). Currently, there are no studies related to Hippo signaling in temporo-mandibular joint function and disorders available. Yet, in a tissue subjected to extreme mechanical loads and stresses such as the TMJ, the Hippo pathway is a prime candidate pathway related to TMD etiology because of its involvement in the cellular response to shear stress, mechanical strain, extracellular matrix stiffness, and mechanotransduction (Dupont et al. 2011, Yu and Guan 2013, Codelia et al. 2014, Ma et al. 2019, Cobbaut et al. 2020). Additional evidence links the two key Hippo transcriptional regulators YAP and TAZ to neuropathic pain (Xu et al. 2016).

The Hippo pathway achieves most of its downstream effects by inhibiting the nuclear functions of its transcriptional co-activators Yes-associated protein (YAP) and TAZ, which display both redundant and non-overlapping functions (Plouffe et al. 2018). Linking the Hippo pathway to inflammation, monomethylation of YAP lysine 494 by the SET domain containing methyltransferase SETD7 is critical for its cytoplasmic retention (Oudhoff et al. 2013). In previous studies it has been demonstrated that the SET containing methyltransferase SETD7 is a pro-inflammatory enzyme that is upregulated during inflammation (Francis et al. 2018). Previous studies also have established that SETD7 mediates the expression of pro-inflammatory genes such as CCL2, TNF-α, and IL-8 and increases the recruitment and stability of NF-KB (Li et al. 2008; Ea and Baltimore, 2009). Antagonistic signaling between YAP/TAZ and NF-κB is involved in the regulation of osteoarthritic cartilage degradation (Deng et al. 2018). In the experiments above, using a CFA treatment rat model, it was demonstrated that the Hippo pathway effector YAP is downregulated under inflammatory conditions, resulting in reduced nuclear localization (FIG. 3) and inability to regulate downstream gene expression in nuclei.

To further understand the role of the Hippo/YAP pathway in the inflammatory processes that lead to TMJ degeneration and pain the SETD7 inhibitor PFI-2 was examined (Barsyte Lovejoy 2014). PFI-2 restored the proliferative potential of inflammation-compromised cells, inhibited cell senescence, reversed inflammatory cell infiltration in tissue implants, and promoted extracellular matrix remodeling (FIG. 4). On tissue sections and in terms of gene expression, the data suggest that PFI-2 treatment returned inflammation damaged TMJ tissues to a near-reversed physiological state, including reduced tissue swelling and inflammatory cytokine expression (FIG. 4). Therefore, a set of in vitro studies are performed to ascertain the inflammatory conditions affect TMJ chondrocytes and synovial stem cells by modulating Yap activity through SETD7. Additional in vitro studies are performed to understand the contribution of the Hippo/YAP pathway toward inflammatory TMD pain via dysregulation of neuropeptide expression and on condylar cartilage homeostasis and cartilage marker genes. A potential role of YAP in the regulation of chondrocyte differentiation via SOX9 has been described in previous studies (Deng et al. 2016, Goto et al. 2018). Together, these studies will lead to a comprehensive mechanistic analysis of the function of Hippo/YAP signaling in inflammatory TMDs and provide a first therapeutic avenue to counter its deleterious effects on TMD pain and condylar degeneration.

As a first step, the expression and localization of SETD7 and YAP are examined in RCs and RSSCs at the mRNA and protein level under physiological and inflammatory conditions. RCs and RSSCs are cultured with or without TNF-α for 6, 12, and 24 hours. Proteins and mRNAs from cultured cells are isolated for RT-PCR and Western Blot analyses. The localization of SETD7 and YAP in relationship toward each other is visualized by immunofluorescence, and the methylation levels and distribution of YAP in nuclei and cytoplasm are determined by Western blotting using anti-SETD7 and anti-YAP antibodies (Abcam). To determine whether YAP and SETD7 interact, co-immunoprecipitation assays is performed, and the degree of interaction between SETD7 and YAP is compared between TNF-α-treated cells and control cells (Ito et al. 2011).

To determine whether inflammatory conditions affect YAP activity through SETD7, and PFI-2 inhibits SETD7-mediated YAP methylation, the status of YAP protein methylation is examined using a modified in vitro methylation assay (Ea and Baltimore, 2009). Nuclear extracts from control and TNF-α treated cells, and recombinant YAP protein (Abcam) are incubated with recombinant SETD7 protein (Active Motif) in the presence or absence of PFI-2 (10 μM) in a reaction buffer containing 1 μM ³H-labeled SAM (Amersham Pharmacia Biotech). Proteins are separated on SDS-PAGE gels and transferred onto an Immobilon-P membrane. The membrane is sprayed with EN3HANCE (NEN) and exposed to Kodak XAR film overnight or detected by anti-methylation antibody (Abcam) for Western Blotting (Francis et al. 2019).

PFI-2 Inhibits Inflammatory Phenotypes Through YAP-NF-κB Interactions. Based on the above results, PFI-2 inhibited the expression of inflammatory cytokines, released fibrous adhesion and reduced inflammatory pain in TMDs (FIGS. 2, 3, and 5). These results also revealed that SETD7 and YAP are expressed in RSSCs and localized in the nucleus (FIG. 3). Inflammatory conditions enhanced SETD7 expression, while YAP nuclear translocation was reduced (FIG. 3). Blocking or knocking down SetD7 increased YAP nuclear translocation and decreased inflammatory gene expression (FIG. 3), indicating that both SETD7 and YAP are involved in gene regulation under inflammatory conditions. NF-kB is a pivotal inflammatory mediator Reciprocal inhibition of YAPI/NF-kB has been reported to regulate osteoarthritis (Deng et al. 2018). In the present study, the involvement of YAP-NF-kB in PFI-2 inhibited inflammation is determined.

To understand the molecular mechanism underlying PFI-2 regulated inflammatory gene expression, YAP and p65 gene knockdown experiments are performed. RSSCs are cultured under inflammatory conditions in the absence or presence of PFI-2 as described above. These cells are transfected with p65 siRNA or YAP siRNA. The expression of inflammatory genes including pro-inflammatory cytokines (IL-1β, IL-6, and TNF-α, adhesion molecules (CDH11, IGβ1, ICAM and Vinculin), and neuropeptides (CGRP, NPY and SP1) is assessed by RT-PCR and Western blot analysis. To test direct regulation of NF-KB and YAP on the expression of these genes, inflammation gene promoter-luciferase reporter constructs are generated by subcloning 3000 bp promoter regions into the pGL-3 luc reporter plasmid (Promega, Madison, WI). The neuropeptide and inflammatory promoter-luciferase reporter constructs are co-transfected with p65 siRNAs or YAP siRNA into RSSC cells. Luciferase activity is detected in protein extracts using a Dual-luciferase reporter system (Promega, Madison, WI) as described previously (Luan et al. 2010). Successful transfection is demonstrated by monitoring p65 and YAP expression levels. These studies address the question whether PFI-2 enhances YAP function through NF-kB inhibition, and in turn regulates inflammatory cytokines (IL-1β, IL-6, TNF-α, adhesion molecules (CDH11, ITGβ1, ICAM) and neuropeptides (CGRP, NYP1 or SP) gene expression.

The effect of PFI-2 treatment on fibrous adhesion in TMD documented in the above data prompted further studies to assess the ability of PFI-2 treatment to regulate cell adhesion. For this purpose, in vitro wound healing model was generated based on an inflammatory cell culture system in the presence or absence of PFI-2. These cells are transfected with p65 siRNA or YAP siRNA. Blocking antibodies for integrin β1 (Millipore), a5 (Abcam), cadherin 11 (Sigma), Fibronectin (Abcam), and Vinculin (Abcam) are used to determine the molecule(s) contributing to PFI-2-regulated extracellular matrix and/or intercellular adhesion. Wounds are generated by a pipet tip after RSSC cells reach confluence. The healing process is recorded under microscope for 2 days. To examine integrin-mediated adhesion strength between cells and ECM, RSSC cells are seeded onto collagen type I coated glass coverslips, while cadherin-, Vinculin-and Fibronectin-mediated adhesion strength of intercellular adhesion are examined with donor RSSC cells seeded on top of a confluent accepter RSSC monolayer on a glass coverslip at ratios of 1:1. Cells are cultured for 15, 30, and 60 minutes at the bottom of a Windkessel chamber (Verrerie Carouge, Geneva, Switzerland) which is used to produce a constant flow of culture medium. After flow wash, attached cells are fixed and quantified in three randomly chosen 40× fields with an inverted microscope. These studies provide quantitative measures for the effect of PFI-2 on cell-cell and cell-matrix interaction in inflamed cell populations.

PFI-2 Restores Chondrocyte Function through YAP-SOX Family Intermediaries: YAP1 regulates multiple steps of chondrocyte differentiation by promoting early chondrocyte proliferation and upregulating SOX6 expression while inhibiting subsequent chondrocyte differentiation and downregulating SOX9 expression (Deng et al. 2016; Goto et al. 2018). The results reported above revealed that inflammation affected YAP1 expression pattern in the condylar growth plate. PFI-2 treatment restored condyle growth plate structure and function and re-localized YAP1 and SOX9 proteins that were altered in response to treatment with inflammatory mediators (FIG. 3)

To understand the molecular mechanism underlying PFI-2 mediated regulation of SOX expression through YAP1, ChIP-PCR experiments are performed. RC cells are cultured under inflammatory conditions in the absence or presence of PFI-2 as described above, and transfected with either YAP siRNAs or YAP overexpression vector Ad-h-YAP1 (Vector Biolabs). The occupancy of YAP1 on the SOX6 and SOX9 promoter TEAD binding sites is examined using ChIP-PCR (Francis et al. 2019). To test direct regulation of YAP1 on SOX gene expression, SOX promoter-luciferase reporter constructs are generated, and luciferase activity detected as described above.

To determine the effect of PFI-2-YAP on chondrocyte proliferation, RC cell growth rates are measured using the MTT cell proliferation assay and BrdU incorporation (Luan et al. 2006). RC proliferation potential at the single cell level is examined by measuring cell colony formation units as described previously (Luan et al. 2006). To further determine the mechanisms affecting RC proliferation, cells subjected to the inflammation culture system are evaluated using senescence-associated beta galactosidase (SA-β gal) assays for senescence (Abcam, Cambridge, UK), and Annexin V staining for apoptosis (BD Biosciences, La Jolla, CA). The expression of senescence-or apoptosis-related genes including cyclin D1, cyclin E, P53, P21 and P16 is determined by qRT-PCR and Western blotting.

To determine the effect of PFI-2 on chondrocyte differentiation and cartilage mineralization under inflammatory conditions, chondrocytes are cultured in two different models, monolayer and pellet culture. For monolayer culture, RCs are plated at a density of 2×10⁴cells/cm², while for pellet culture, the initial number of RCs is 4×10⁶ cells/pellet. Cells are cultured for a 7-10-day period to assess differentiation and a 15-20-day period to assess mineralization. RNAs and proteins are isolated at each time point to examine chondrocyte phenotype and differentiation marker expression levels, including Col 2a1 and Col10a1. Safranin O staining is performed to detect the proteoglycan content and Alizarin Red staining to determine the mineralization status in the culture environment (Luan et al. 2006).

Determination of the Role of YAP Signaling in TMJ Homeostasis in Vivo: To further confirm the physiological role of YAP in TMJ tissues, TAZ flox YAP flox double conditional knockout mice are utilized (Jackson Laboratory). These mice are cross-bred with Col1A1CreERT2 or Col2CreERT mice. To generate tamoxifen (TM)-inducible and mesenchymal or cartilage-specific TAZ/YAP knockout mice (Col1-CreER; TAZ/YAPfl/fl or Col2-CreER; TAZ/YAPfl/fl), Tg(Colla1-cre/ERT2)1Crm/J or Tg(Col2a1-cre/ERT)KA3Smac/J mice (Jackson) are crossed with TAZ flox YAP flox mice. Six weeks old Col1-CreER; TAZ/YAPfl/fl or Col2-CreER; TAZ/YAPfl/fl mice receive intraperitoneal injections of tamoxifen (Sigma-Aldrich, 0.1 mg/g of body weight) daily for 5 consecutive days. Routine genotyping of mouse tail DNA is confirmed by RT-PCR. Col1- and Col2-CreER; TAZ flox YAP flox mice are used as negative controls. Mice from each of these four groups receive saline or CFA injections into the TMJ to generate inflammatory TMD (FIG. 1). Changes in tissue structure and gene expression as well as pain behavior from these mice are analyzed as described elsewhere herein.

Investigation into the involvement of other signaling pathways, such as the Wnt or NF-κB pathways, is ongoing, since SETD7 also modifies β-Catenin and P65 protein through methylation. To identify individual transcriptome changes of unique YAP mutant and control TMJ subpopulations on a single cell level under physiological and inflammatory conditions, single cell analysis are conducted.

### Example 3

### PFI-2 Small Molecule-Based Engineering to Alleviate Hippo Pathway-Mediated Inflammatory TMJ Destruction and Diminish TMD Pain

The clinical treatment of TMDs currently focuses on three distinct approaches, depending on TMD severity in terms of pain and functional limitations as well as the expertise of the medical/dental practitioner (Dashnyam et al. 2018): (i) noninvasive treatment modalities including physical therapy, behavioral intervention, and pharmacologic approaches, (ii) minimally invasive treatment modalities including joint space flushing and draining to remove inflammatory mediators and enhance lubrication by means of needle injections and arthrocenteses, and (iii) surgical approaches including invasive intervention and alloplastic replacement in advanced or chronically degenerated TMDs with limited mouth opening (Willard 2014). While surgical approaches for TMD treatment have a history of uncertain outcomes irrespective of their highly invasive nature, noninvasive forms of treatment often will not yield the immediate turnaround in terms of pain or function that the TMD patient community is hoping for. As a consequence, intra-articular drug and biomolecule delivery via intra-articular injections emerges as the method of choice for the restoration of TMJ structure and function. However, currently there are no agents known that will reverse TMDs to a healthy state (Dashnyam et al. 2018). Even if such an agent was identified, ideal molecules would clear rapidly and as such require repeated injections, resulting in complications such as infection, fibrosis and joint damage. To harness the advantages of minimally invasive TMD treatment and address its current limitations, a biomaterial-and exosome-assisted delivery system that administers the PFI-2 small molecule probe to the diseased TMJ via intra-articular injection has been developed.

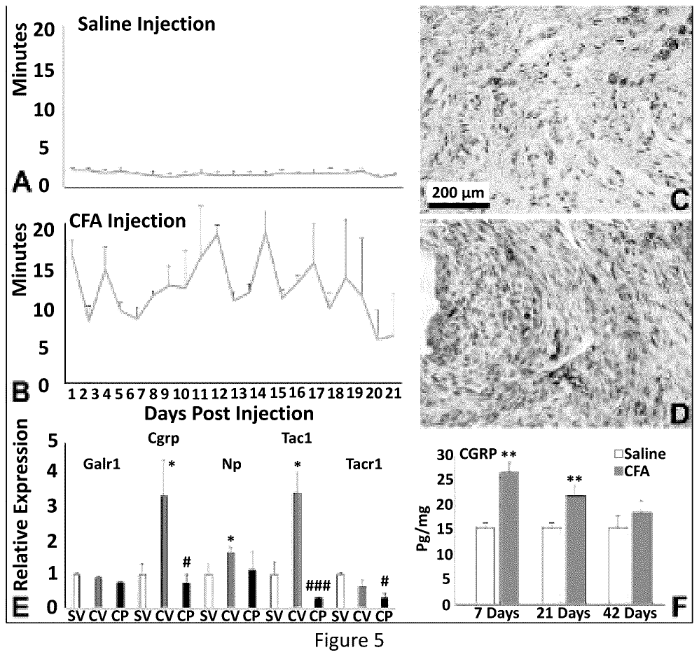

Rat synovial mesenchymal stem cells (RSSCs) were cultured and examined for SETD7 nuclear localization. Treatment of RSSCs with TNF-α increased nuclear localization of SETD7 and expression of SETD7 in the cytoplasm (FIG. 5A, FIG. 5B, and FIG. 5G), however treatment with PFI-2 blocked the effect of TNF-α (FIG. 5C and FIG. 5G). Treatment of RSSCs with TNF-α resulted in decreased nuclear localization of YAP, which was rescued by SETD7 siRNA (FIG. 5D-FIG. 5F and FIG. 5H). Knockdown of other proteins, including YAP and proteins upstream of YAP, revealed that knockdown of P65 and SETD7 reduced the expression of inflammatory cytokines, while knockdown of YAP resulted in an increase of inflammatory cytokines. Western blot analysis of protein methylation in whole cell lysates and YAP expression in the nucleus demonstrated that PFI-2 treatment reduces methylation and YAP nuclear localization in RSSCs treated with TNF-α (FIG. 5I and FIG. 5J). Examination of YAP binding to the SOX9 promoter TEAM binding site by chip assay demonstrated that YAP1 knockdown reduces relative YAP1 enrichment on the SOX9 promoter while the while the expression of SOX9 was increased, as demonstrated by RT-PCR (FIG. 5L and FIG. 5M). Additional changes in neuropeptide expression were observed upon YAP knockdown in RSSCs, including upregulation of CGRP, galanin, and TAC, while NPY was downregulated.

Further examination of RSSCs treated with TNF-α, with the addition of PFI-2, demonstrated that PFI-2 rescues TNF-α inflammatory phenotypes and gene dysregulation. Labeling of RSSCs with BrdU demonstrated a decrease in BrdU-positive actively proliferating cells treated with TNF-α, while PFI-2 treatment restored cellular proliferation (FIG. 6A-FIG. 6C). β-gal staining of senescent cells demonstrated that TNF-α induces cellular senescence, which was similarly inhibited by PFI-2 treatment (FIG. 6D-FIG. 6F).

In vivo analysis was examined by subcutaneous implantation of collagen sponges coated with PBS, TNF-α, or TNF-α and PFI-2. It was observed that cell infiltration was increased by TNF-α, while PFI-2 reversed this effect and promoted ECM remodeling(FIG. 6G-FIG. 6I). Cultured condyle treated with BSA, IL-6, or IL-6 plus PFI-2 were immunostained with IL-18, demonstrating an increase in thickness of fibrous and proliferative zones upon exposure to IL-6. PFI-2 treatment reversed the swelling induced by ILJ-6 and also inhibited the expression of IL-18 (FIG. 6J-FIG. 6L). qRT-PCR analysis of select inflammatory genes demonstrated that TNF-α treatment increased SETD1, IL-6, and MMP2 expression, which was inhibited by treatment with PFI-2 in a dose-dependent manner (FIG. 6M), which was confirmed by Western blotting (FIG. 6N).

Three different carrier materials for the injection of PIF-2 are compared, including a (i) collagen gel (Dangaria et al. 2011), (ii) a TMJ disc hydrogel prepared from porcine TMJ discs as a tissue specific carrier, and (iii) MSC-derived exosomes prepared from rat synovial mesenchymal stem cells and chondrocytes as a cell-specific scaffolding material. The efficacy of PFI-2 delivery using various carrier scaffolds on the regeneration of damaged TMJ tissues and altered TMJ function are examined.

Delivery of the SETD7 inhibitor PFI-2 via biomaterials-and exosome-assisted delivery systems relieves TMJ pain and alleviates TMD inflammatory degradation in an inflammatory TMD animal model.

Preparation and characterization of PFI-2-releasing collagen hydrogel: to generate a PFI-2-releasing collagen hydrogel, commercially available bovine collagen type I hydrogel (Col I) in injectable form (Advanced Biomatrix) is used. PFI-2 is mixed with Col I gel (10 mg/ml, pH 7) at a concentration of 10 μM based on preliminary studies. The mixture is incubated at 4° C. overnight to promote the interaction and formation of amine bands between PFI-2 and collagen. To characterize the release profile, PFI-2-Col I is immersed in a culture medium containing bovine serum at a ratio of 1:1 and incubated in a rotary shaker at 37° C. Samples are collected, and PFI-2 release is determined at 5 time points over a 20 day period using HPLC. The cumulated release rate is calculated by the amount of PFI-2 released from the PFI-2-Col I gel as percentage of total incorporated PFI-2. Attachment of cells to the hydrogel was examined by coating a culture dish, which was seeded with RSSCs (FIG. 7N).

Preparation and Characterization of PFI-2-deCM Hydrogel: to Prepare TMJ tissue specific, de-cellularized extracellular matrix (dECM) hydrogel from TMJ discs as a carrier for PFI-2 delivery, fresh porcine TMJ discs are harvested from pig jaws. The TMJ discs are minced into small pieces followed by 4 cycles of freezing and thawing. These samples re treated with trypsin, nuclease solution, and Triton® X-100 for decellularization. The decellularized TMJ disc tissue are then lyophilized to obtain powdered ECM. TMJ disc dECM (150 mg) is digested with pepsin to form a flowable viscous solution. This solution is neutralized by 0.1 M sodium hydroxide. The entire procedure is performed in an ice bath to avoid gelation of the TMJ disc dECM. To determine the decellularization efficiency, the decellularized TMJ discs are processed for paraffin sections which are stained with hematoxylin and eosin. DNA quantification in the decellularized tissues is performed using the PicoGreen DNA assay kit (Life Technologies, Inc. Carlsbad, CA) after DNA extraction and precipitation. To characterize the TMJ disc dECM hydrogel, the morphology of the hydrogel is examined using SEM (FIG. 7A). The rheological characteristics of TMJ disc dECM hydrogels are measured by temperature-sweep rheological experiments (FIG. 7B). Gelation kinetics and shear moduli of the TMJ disc-derived hydrogels are evaluated by parallel-plate rheology (FIG. 7C and FIG. 7D). The crossover point of storage modulus and loss modulus in the time sweep mode is used to indicate the gelation time (Liang et al. 2020). Proliferation of cells in dECM hydrogel was examined, and found to be higher than that of Col I hydrogel (FIG. 7E and FIG. 7F). Injection of dECM hydrogels was examined by subcutaneous injection (FIG. 7G). After one hour, the implant was devoid of cells (FIG. 7H), while multiple cells had migrated to the implant after 7 days, as demonstrated by H&E staining (FIG. 7I). Attachment of RSSCs to dECM hydrogel was examined as described in the PFI-2/Col I section above, and yielded greater surface coating, indicating the hydrogel specifically targets RSSCs (FIG. 70). PFI-2 loading and controlled release properties are monitored as described in the PFI-2/Col I gel preparation section above.

Preparation and Characterization of PFI-2 Loaded Exosomes: to Develop an exosomal delivery system directly targeting the TMJ synovial membrane or condyle for PFI-2 application in TMD treatment, exosomes are isolated from cultured rat synovial mesenchymal stem cells (RSSCs) or rat condylar chondrocyte cultures (Luan et al. 2006; Dangaria et al. 2011). The cells are cultured until they reach 80-90% confluency, and then incubated in the exosome conditional medium (CM) supplemented with 0.5% exosome-depleted fetal bovine serum (Van Deun et al. 2014; Schindler et al. 2019). Following collection of the medium, cells are trypsinized, and cell viability measured to confirm a minimum viability of 94% and avoid potential apoptotic bodies during exosome isolation. The collected CM is concentrated, and the concentrated CM is centrifuged to remove the larger vesicles and finally be pelleted. The successfully isolated exosomes are suspended in PBS and stored at −80° C.

To characterize the exosomes, nanoparticle diameter and concentration are assessed using the qNano Nanoparticle Characterization System (IZON Science, FIG. 7J and FIG. 7K). For transmission electron microscopy (TEM), exosomes are absorbed to a Formvar-carbon coated electron microscope grids (Leica) and then contrasted with 2% uranyl acetate. Exosome size and morphology are observed using a JEOL JEM-1200 electron microscope (Jokisaari et al. 2019). To detect exosome specific markers (CD63, CD-81, Lamp2, Alix; Santa Cruz Biotechnology), Western blot analysis is carried out as described previously (Dangaria et al. 2013; FIG. 7L).

To prepare PFI-2 loaded exosomes, PFI-2 is added to exosomes (200 μg/ml of total protein, 20% of loading capacity) at a concentration of 10 μM and then ultrasonicated. The sonicated exosomes are centrifuged to remove aggregates and un-encapsulated PFI-2, and further centrifuged to pellet the PFI-exosomes. All supernatants are collected to determine PFI-2 encapture efficiency rate (EE%) using the formula [(total PFI-2 added-free non-entrapped PFI-2)/PFI-2 added]×100. Fluorescence uptake by exosomes was additionally examined in order to verify loading (FIG. 7M). To determine PFI-2 controlled release from the PFI-2-loaded exosomes, the exosomes are transferred to dialysis tubes (molecule weight cut off 14 kDa) and incubated in PBS containing 10% FBS in a rotary shaker. PFI-2 release is determined at five time intervals over 20 days. The cumulated release rate is calculated by the amount of PFI-2 released from the exosomes as percentage of total PFI-2.

Injection of PFI-2-Col I, PFI-2-decm Hydrogel and PFI-2-exosome solution into the TMJ: Four groups (SV, SP, CV, and CP) receive injections of the saline plus carrier, saline plus PFI-2 carrier, CFA plus carrier, or CFA plus PFI-2 carrier, respectively. The TMJ injection procedure is described in FIG. 1. Forty-eight hours after the injection of saline or CFA solution, PFI-2 (10 μM) in a carrier, or the carrier alone, is injected every two days, or once within 10 days. The injection times and effect duration of PFI-2 on both inflammatory degeneration and pain are optimized.

Delivery and transfection efficiency of the controlled-release molecules are determined using fluorescence-labeled PFI-2 hydrogels or PFI-2 exosomes. PFI-2-Col, PFI-2-dECM hydrogels and PFI-2-exosomes are labeled using the VivoTag-S 680 Protein Labeling Kit (PerkinElmer) according to the manufacturer's instruction. Five days, 10 days, and 4 weeks after injection, TMJs and surrounding tissues are fixed and embedded in OCT to cut frozen sections, or frozen for RNA and protein extraction. The frozen sections are observed under a fluorescent microscope to locate VivoTag-S fluorochrome. The sections are also stained with fluorescently labeled antibodies against CD40 for fibroblasts, CD31 for endothelial cells, and CD11b for myeloid cells. Locations and types of labeled cells are identified by co-localization of VivoTag-S fluorochrome and fluorescence-labeled antibodies. To assess delivery efficiency, quantitative RT-PCR is performed for the expression of Yap downstream genes including Cyclin D1 and CTGF. Comparison of fluorescence intensity, and gene expression between experimental and control animals indicates successful molecule delivery and functional efficiency.

Bone volume measurements, mechanical properties, and histomorphometric measurements of TMJ layer dimensions greatly change in response to inflammatory conditions, and after treatment with the three PFI-2 delivery modalities. Specifically, inflammatory conditions alter bone volume, reduce mechanical properties, and affect the thickness of functional condyle layers, while PFI-2 delivery restores outcomes to near-physiological values. Condyles and discs from all five groups are subjected to a rigorous mechanical, morphometric, and bone parameter analysis. All measurements are performed independently by two different observers, and biostatical analysis is performed. In addition, the effect of PFI-2 on pain is assessed after 10 days and 4 weeks as described.

Injectable collagen and dECM hydrogel are used as carrier materials for PFI-2 controlled release. These PFI-2 loaded materials have a burst release of approximately 40% total loading over the first 24 hours and reach a state of steady release four days after the injection. A more sustained release for PFI-2 treatment, is achieved by collagen or dECM nanoparticles using an emulsion technique. The nanoparticle-based delivery system provides an extended release of PFI-2 in TMJ tissues.

PEAP behavior of animals was investigated after administration of saline, CFA, or CFA and a PFI-2 carried by either a Col I hydrogel or a dECM hydrogel. Shortening of time spent in the dark half of the chamber by CFA administration was significantly rescued by administration of either PFI-2 hydrogel, but was far more significant with the dECM hydrogel (FIG. 7P and FIG. 7Q).

The methods and materials employed are described herein.

Macroscopic analysis and bone quality comparison. At two time points, ten days and 4 weeks after injection, ten TMJ discs and corresponding condyles from each of the four treatment groups, (control, CFA treatment, PFI-2 control, CFA+PFI-2 group treatment) with different carriers are dissected. The condyles are scanned using a desktop cone-beam microCT scanner Scanco model 40 (Scanco), and volumetric measurements and bone density are measured using a TeraRecon software package (Lu et al, 2013).

Mechanical testing. Hardness and Young's Modulus measurements of condyles are assessed using a microindentation head on a 5948 MicroTester (Instron, Norwood, MA). Young's modulus is calculated based on indentor load and radius as described by Li et al. 2006. Experimental groups and controls are designed as described above.

Histomorphometric Assessment of the Effect of PFI-2 on the Regeneration of TMJ tissues. For histomorphometric analysis, the condyles as well as discs and synovial membranes are prepared for paraffin sections, and histomorphometric data including thickness and histology, are compared. Specifically, 10 adjacent mid-sagittal section through ten left condyles/group are prepared from each of the four treatment groups described above. Sections are dehydrated and stained with H&E, and micrographs are processed for morphometry. Micrographs are analyzed to assess the thickness of the fibrous articular layer, the proliferative zone, and the hypertrophic cartilage. Linear measurements are obtained from the posterior articular region, the posterosuperior growth region, and the superior articular region as described by McNamara and Carlson (1979). Additional data of central and lateral TMJ disc diameter are obtained from each micrograph. In addition, collagen fiber length and orientation are visualized on each section using polarization microscopy, and measurements are compared among groups as described.

Protein and mRNA data analysis. The regenerated tissues are also collected for mRNA and protein extraction. In addition, the expression of inflammatory genes as well as osteogenesis and chondrogenesis-related genes is examined using RT-PCR and Western Blotting for key extracellular matrix genes, neuropeptides, and Hippo pathway intermediaries. This set of data allows assessment of the potential of PFI-2 for TMJ regeneration.

Biostatistics and Power Analysis. the SAS Software 9.4 Is Used for statistical and power analysis. Data are described as mean±standard deviation. Comparisons are analyzed using one-way ANOVA followed by Tukey HSD post hoc tests with a significance level of p <0.05. Animal numbers are estimated using the SAS POWER procedure. A power of 0.8 and a significance of 0.05 is achieved for 4 different parameters per study with 36 in vivo and in vitro samples (n=36 samples or 18 rats: 8samples for μCT and histology, 10 for RNA analysis, 8 for mechanical testing, and 10 for protein extraction).

Sex as a biological variable. Temporo-mandibular joint disorders have a 2:1 prevalence in women compared to men (Bueno et al. 2018). To assess potential gender differences between male and female animals, 3 male and 3 female animals of each group are analyzed for differences in pain behavior, bone parameters, histomorphometry, and gene expression. Further in-depth studies are conducted when parameters turn out to reveal significant evidence of gender differences.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

The disclosures of each and every patent, patent application, and publication cited herein are hereby incorporated herein by reference in their entirety.

1. A method of treating or reducing pain in a subject in need thereof, comprising administering to the subject a therapeutically effective amount of a composition comprising PFI-2 or a derivative, prodrug, or pharmaceutically acceptable salt thereof.

2. The method of claim 1, wherein the pain is associated with one or more inflammatory diseases or disorders.

3. The method of claim 2, wherein the one or more inflammatory disease or disorders are one or more temporo-mandibular joint and muscle disorders (TMDs).

4. The method of claim 3, wherein the composition is injected directly into the temporo-mandibular joint (TMJ) or the surrounding tissue.

5. The method claim 1, wherein the composition comprises one selected from the group consisting of a hydrogel, a nanoparticle, an exosome, and combinations thereof.

6. The method of claim 5, wherein the hydrogel is a collagen hydrogel or extracellular matrix (ECM) hydrogel.

7. The method of claim 6, wherein the ECM hydrogel is a decellularized ECM (dECM) hydrogel.

8. The method of claim 6, wherein the collagen hydrogel is a Type I collagen (Col I) hydrogel, with Col I at a concentration of about 10 mg/mL.

9. The method claim 6, wherein the concentration of PFI-2 in the hydrogel is about 20 μM.

10. The method of claim 5, wherein the nanoparticle is a Col I hydrogel nanoparticle or a dECM hydrogel nanoparticle.

11. The method of claim 5, wherein the exosome is derived from a mesenchymal stem cell (MSC).

12. The method of claim 11, wherein the MSC is a synovial MSC.

13. A composition comprising PFI-2 or a derivative, prodrug, or pharmaceutically acceptable salt thereof and one or more selected from the group consisting of a hydrogel, a nanoparticle, an exosome, and combinations thereof.

14. The composition of claim 13, wherein the composition is formulated for injection.

15. The composition of claim 13, wherein the hydrogel is a collagen hydrogel or an ECM hydrogel.

16. The composition of claim 15, wherein the collagen hydrogel is a Col I hydrogel.

17. The composition of claim 15, wherein the ECM hydrogel is a dECM hydrogel.

18. The composition of claim 13, wherein the nanoparticle is a Col I hydrogel nanoparticle or a dECM hydrogel nanoparticle.

19. The composition of claim 13, wherein the exosome is derived from an MSC.

20. The composition of claim 19, wherein the MSC is a synovial MSC.
